(19) United States
(12) Patent Application Publication
(10) Pub. No.: US 2010/0168831 A1
(43) Pub. Date: Jul. 1, 2010

(54) Implantable clip-on micro-cuff electrode for functional stimulation and bio-potential recording

(75) Inventors: Naga Srinivas Korivi, Baton Rouge, LA (US); Pratul K. Ajmera, Baton Rouge, LA (US)

Correspondence Address: Adams and Reese LLP, 1221 McKinney Street, Suite 4400, Houston, TX, 77010, US

(73) Assignee: Board of Supervisors of Louisiana State University and Agricultural and Mechanical College, Baton Rouge, LA (US)

(21) Appl. No.: 12/653,476

(22) Filed: Dec. 15, 2009

Related U.S. Application Data
(60) Provisional application No. 61/203,868, filed on Dec. 30, 2008.

(51) Int. Cl. A61N 1/05; B32B 37/26

(52) U.S. Cl. 607/118; 264/134

(57) ABSTRACT

A nerve cuff electrode with a clip-on design feature is provided for the stimulation of nerves and nerve fibers and the recording of bio-potentials from nerves and nerve fibers. The nerve cuff includes an aperture having a longitudinal slit, a pinch hinge having a first arm and a second arm spaced apart from each other and at least one electrode embedded in the inner circumference of the aperture. The longitudinal slit is configured to open when a compressive force is applied to the outer surfaces of the first arm and the second arm whereby the first and second arm are squeezed together. When the compressive force is removed, the spring tension of the cuff returns the longitudinal slit to its normally closed position.

### CROSS-REFERENCE TO RELATED APPLICATIONS

This application claims the benefit of U.S. Provisional Patent Application Ser. No. 61/203,868 filed Dec. 30, 2008 which is expressly incorporated herein in its entirety by reference thereto.

### FIELD

The present invention relates generally to the field of functional electrical stimulation. More particularly, the present invention relates to implantable devices used to send and receive electrical signals to and from nerves and nerve fibers, and the fabrication of such devices.

### BACKGROUND

The developing field of functional electrical stimulation (“FES”) generally involves the application of electrical signals to specific nerves and muscles to induce specific physiological responses. The field has developed to support a variety of applications, including, but not limited to, neuromuscular control, optical prostheses, pain therapy, and morbid obesity treatment. In this field, “cuff” electrodes which may partially or completely encircle the nerve fiber or branch to be stimulated/monitored, are used. The use of FES in such situations provides a minimally invasive, reversible treatment requiring minimal post-operative care as opposed to highly invasive, non-reversible and expensive surgical alternatives.

FES is based on the periodic or constant electrical stimulation of specific nerves, nerve fibers and muscles by a single or multiple set of electrodes, for control and treatment of numerous pathological conditions. Numerous electrode designs have been developed to date for applications involving functional electrical stimulation of nerves and nerve fibers. One of the most commonly employed electrode design is the cylindrical cuff electrode. This design is simple and relatively easy to use for long term use in subjects even when they are moving. One of the other reasons for a general preference for this type of electrode is the geometry of a typical nerve or nerve fiber, considered to be cylindrical from a design perspective. However, there are some problems involved in the use of the cuff design that are not addressed by currently available cuff electrodes. The currently available cuff electrodes must be manually opened, placed on the target nerve and then closed prior to their operation. The cuff opening is typically secured by epoxy, wax or small piece of silicone rubber. This involves the use of intra-operative procedures to secure or seal the cuff opening immediately after implantation. The use of intra-operative procedures to seal the cuff opening becomes significantly more complicated when the cuff electrodes are small in dimensions. Therefore, there is a need for an electrode design that can preserve the advantages of a cuff structure, while overcoming the problem of securing the cuff and cuff opening.

A further problem with available cuff electrodes is that they are not suitable for small anatomies, i.e. smaller nerves and nerve fibers. Such small nerves and nerve fibers need smaller cuff electrodes, with cuff dimensions often in the micrometer scale. For example, cuff electrodes of about 250-450 micron inner diameter are needed for rat celiac and splanchnic nerves. It is difficult to open and close regular cuff structures as they approach such small sizes. Therefore, there is a requirement for .a cuff electrode design that can effectively address the stimulation of small diameter nerves and nerve fibers.

Another problem with present cuff electrode technology is the lack of sufficient designs that ensure a complete closure of the cuff around the nerve or nerve fiber. A completely closed cuff ensures that there is almost a fixed amount of extracellular fluid trapped around the nerve by the cuff. Extracellular fluid can conduct electricity. A varying amount of extracellular fluid may surround the nerve in the case of partially open cuff. This may cause electrical noise and is not desirable. This is especially important for applications involving the reading of bio-potentials from nerves, for which extremely small electrical noise is desirable.

Existing cuff electrode designs are typically based on the assembly of discrete components including cylindrical tubing serving as the cuff, and metal wires serving as electrode leads. The electrode wires are inserted into the cuff and secured by means of epoxy. This reduces the robustness of the device, as the electrode wire and cuff wall interface is vulnerable to pulling forces acting on the wire. Detachment of the wire from the cuff results in device failure, requiring another surgical re-implantation of the electrode. A cuff electrode design that provides for a robust electrode wire-cuff interface is very important.

Thus, currently available nerve cuff electrodes suffer from a number of drawbacks, including:

1. Once maneuvered about the targeted tissue, cuffs are difficult to close.

2. Cuffs that can be closed completely are required to be crimped or glued shut after placement, which increases the potential for damage to the tissue, which could result in paralysis or death of the patient/animal subject.

3. Cuffs that cannot be closed completely are prone to electrical interference or “cross-talk” from nearby tissues or other nearby physiological ambients.

4. Techniques such as epoxy gluing, wire securers, and suture stitching used to secure many current electrodes to the target tissue also increase the possibility of trauma to sensitive nerve fibers.

5. The main body of the cuff is comprised of different materials/components which may be attached to each other by adhesive. Such components, particularly the electrode wire-cuff interface, can become detached, resulting in device failure.

6. Smaller anatomical requirements are not addressed, as the existing electrode designs for micro-scale diameter nerves typically require complicated manufacturing processes involving specialized photolithographic equipment or other equipment such as physical/chemical deposition systems and plasma-based etchers.

### SUMMARY

According to an exemplary embodiment of the present invention, a nerve cuff is provided that includes an aperture having a longitudinal slit located opposite a pinch hinge having a first arm and a second arm, the first arm spaced apart from the second arm and at least one electrode embedded in the interior circumference of the aperture, wherein the longitudinal slit may be opened when a compressive force is applied to the outer surfaces of the first arm and the second arm by squeezing the first arm and second arm together.

### BRIEF DESCRIPTION OF THE DRAWINGS

FIG. 1 is an isometric view of a nerve cuff that incorporates a pinch hinge, according to an exemplary embodiment of the present invention.

FIG. 2 is an isometric x-ray view of a nerve cuff that incorporates a pinch hinge, according to an exemplary embodiment of the present invention.

FIG. 3 is a series of three increasingly magnified images of a nerve cuff that incorporates a pinch hinge, according to an exemplary embodiment of the present invention.

FIG. 4 is an image of the longitudinal slit of a nerve cuff that is shown with a gap and incorporates a pinch hinge, according to an exemplary embodiment of the present invention. The gap can be reduced to zero or changed as desired.

FIGS. 5 and 6 illustrate the steps for a method of manufacturing a nerve cuff that incorporates a pinch hinge, according to an exemplary embodiment of the present invention.

FIGS. 7 and 8 illustrate the steps for an alternative method of manufacturing a nerve cuff that incorporates a pinch hinge, according to an exemplary embodiment of the present invention.

FIG. 9 illustrates the steps for yet another alternative method of manufacturing a nerve cuff that incorporates a pinch hinge wherein the method incorporates a single molding step according to an exemplary embodiment of the present invention.

### DETAILED DESCRIPTION

According to an exemplary embodiment of the present invention, a nerve cuff is provided that allows for complete closure and that may be useable in smaller anatomical settings, such as micro-scale diameter nerves. Such an implantable clip-on micro-cuff electrode may incorporate a pinch hinge design feature. The cuff may be opened by applying pressure to the pinch hinge, located directly opposite a longitudinal slit in the cuff. Once opened, continued pressure is applied to the hinge as it is maneuvered into place about the nerve tissue. When released, the spring tension of the pinch hinge returns the cuff to its normally closed position. Because the installer need take no further action to close the cuff, the possibility of damage to the nearby nerve tissue is reduced.

According to an exemplary embodiment of the present invention, a clip-on design of a micro-cuff, which incorporates a pinch hinge, is easier to attach than current commercially available nerve cuffs, thereby significantly reducing the possibility to inflict damage to nervous tissue. The pinch hinge structure enhances the general ease of handling the nerve cuff as it is maneuvered into the desired position, and the clip-on structure is configured such that the cuff electrode remains secured to the nerve in-vivo regardless of the movements of internal organs that may be caused by the physical motion of the animal or person. Techniques such as epoxy gluing, wire securers and suture stitching are not necessary to ensure the cuff remains in place. The possibility of damage to the nervous tissue is thereby significantly reduced.

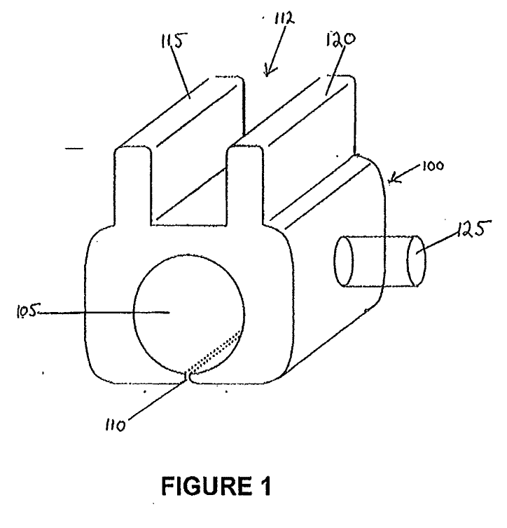

FIG. 1 shows a nerve cuff with an incorporated pinch hinge feature according to an exemplary embodiment of the present invention. A nerve cuff may include a member 100 including an aperture 105 having a longitudinal slit 110 opposite a pinch hinge 112. The hinge 112 includes a first arm 115 and a second arm 120. One or more electrodes and an electrode wire may be embedded in the member. The spring tension of the member 100 holds the longitudinal slit 110 in a normally closed position. The longitudinal slit 110 is configured to open when a compressive force is applied to an outer surface of the first arm 115 and the second arm 120, thereby compacting the arms together and overcoming the spring tension holding the longitudinal slit 110 closed. Continuous compressive force may be applied to the outer surfaces of the first arm 115 and the second arm 120 to maintain the cuff in an open position. Cessation of the compressive force to the outer surfaces of the first arm 115 and the second arm 120 will allow the spring tension in the member 100 to return the longitudinal slit 110 to the normally closed position. A port 125 may provide access to the electrodes by the electrode wire. In some other embodiments, the wires may be directly embedded inside the main body 100. In some other embodiments, the wire access with or without the use of port may be made across the longitudinal opening slit 110 and between the two arms 115 and 120 of the pinch hinge. In some embodiments of the present invention, the member 100 and pinch hinge 112 may be formed from the same material or piece of material. In other embodiments, the member 100 and pinch hinge 112 may be formed from different materials and/or as separate pieces of the same material joined together in the fabrication process.

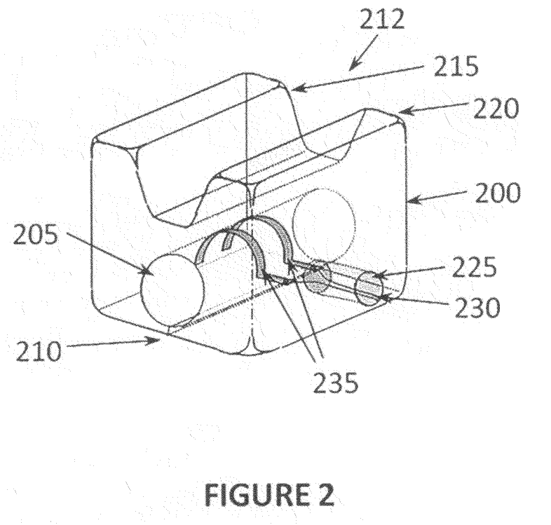

FIG. 2 shows isometric “x-ray” view of a nerve cuff with an incorporated pinch hinge feature according to an exemplary embodiment of the present invention. A member 200 may include an aperture 205 having a longitudinal slit 210 opposite a pinch hinge 212, which includes a first arm 215 and a second arm 220. One or more electrodes 235 may be positioned in the inner circumference of the aperture 205 such that the electrodes 235 are positioned near or in direct contact with a nervous tissue when the cuff is positioned about a nerve and allowed to return to its normally closed position such that the aperture 205 completely encircles the nerve. A port 225 allows access to the electrodes 235 by one or more electrode wires 230, which may be spot welded to the electrodes 235. In some other embodiments, the port 225 may be located between the two arms 215 and 220 of the pinch hinge 212. In some other embodiments, the wires may be embedded directly into the body 200 without use of a port. The electrode wires 230, which may be either coated with an insulation or without insulation, may also be further insulated from each other and the surrounding environment by silicone tubing or other appropriate insulating material, which may define port 225.

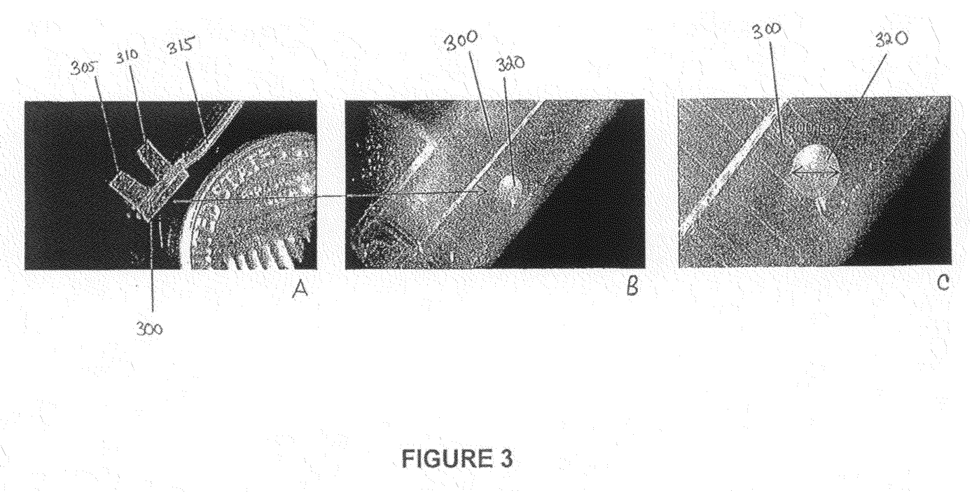

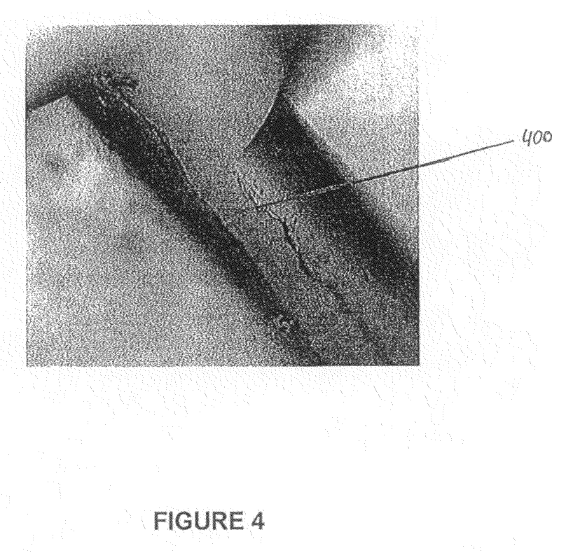

FIG. 3 shows a series of three increasingly magnified views of a nerve cuff that incorporates a pinch hinge feature, according to an exemplary embodiment of the present invention. A member 300 may incorporate a pinch hinge having a first arm 305 and a second arm 310 opposite an aperture 320. The aperture may be as small as approximately 150 micro-meters, according to certain embodiments of the present invention. An insulated wire 315 may carry signals to and from electrodes embedded in the aperture 320. FIG. 4 shows a close up image of a longitudinal slit 400 in an aperture opposite a pinch hinge, according to an embodiment of the present invention. In certain embodiments, the longitudinal slit 400 may be from near 0 to about 100 micrometers across.

According to an exemplary embodiment of the present invention, a relatively simple fabrication process of a micro-cuff provides for the manufacture of a micro-scale diameter cuff that makes proper electrical contact with an enclosed nervous tissue while reducing the potential for trauma that may result from such contact. Existing electrode designs for micro-scale diameter nerves typically require complicated manufacturing processes involving specialized equipment and may involve more than one material and/or subcomponent which are attached to each other by adhesive. In contrast, according to an exemplary embodiment of the present invention, a fabrication process includes micro-molding of biocompatible silicone elastomer as a base, onto which the metal electrodes and additional layers are added via spin-coating. The completed cuffs may be separated by dissolution of the sacrificial layer followed by cutting. According to an exemplary embodiment of the present invention, the hinge and body of the nerve cuff may be formed as a single piece. The use of the same material for the device body and the embedded insulation of the electrode lead wires may result in a simplified fabrication process and allows for enhanced structural robustness and smaller (micro-scale) cuffs. Force tests performed on embodiments of the present invention have shown that the electrode wires can withstand significant values of pulling force (more than typically expected force values), without becoming displaced from the main electrode body.

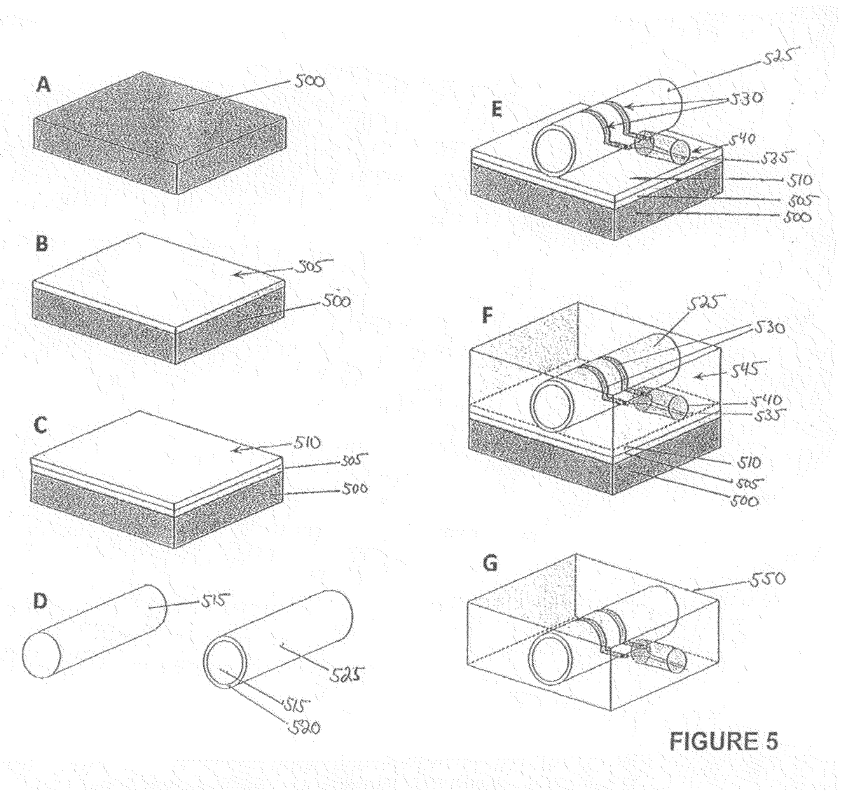

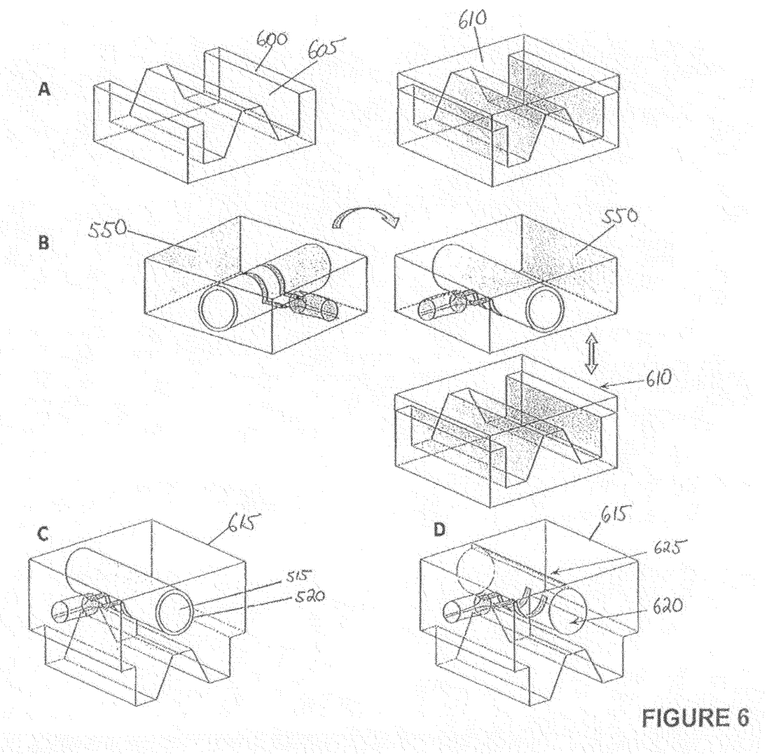

FIGS. 5 and 6 illustrate the steps for a method for fabricating a nerve cuff that incorporates a pinch hinge feature, according to an exemplary embodiment of the present invention. First, a thin flat sheet of plastic or metal is cleaned to be used as the base substrate 500. A commercially available mold release chemical may then be spin-coated onto the substrate to function as a mold release layer 505. The spin coating process may be accomplished by dispensing a specific amount of mold release chemical on the substrate 500 and spinning it horizontally at a specific rotational speed. The spinning operation may be performed by a spin-coater machine. The mold release chemical may be configured such that it may be dissolved in a specific solvent.

After the mold release layer 505 is allowed to solidify at room temperature, a thin layer of silicone 510 which, in certain embodiments, may be medical grade, silicone, is spin-coated onto the solid mold release layer 505. The silicone layer 510 is subsequently cured, said curing being accomplished in some embodiments at a temperature range appropriate for the silicone elastomer in use e.g. 60 degrees centigrade to about 70 degrees centigrade.

Next, a metal wire 515 of a known diameter is coated with a thin layer of the mold release chemical 520. In some embodiments the metal wire 515 may be commercially available copper wire. In some embodiments, the coating 520 may be applied to the metal wire 515 by dipping the wire in the mold release chemical and withdrawing it at a specific rate. The wet mold release chemical layer 520 may be solidified at room temperature.

The coated metal wire 525 may then be placed in conformal contact with the thin silicone layer 510. Two metal foil electrodes 530 may be placed in contact with approximately half the circumference of the coated metal wire 525. In certain embodiments, the metal foil electrodes 530 may be previously cut to size from commercial available materials such as platinum, platinum-iridium, stainless steel or any other metal of choice. The metal foil electrodes 530 may be spot-welded to stainless steel wires 535, which may serve as interconnecting wires from the implanted electrodes 530 to external electronic equipment. The stainless steel wires 535 may surrounded by an insulation layer 540 to protect the wires 535 from the external environment. In certain embodiments, the insulation layer 540 may be medical grade silicone.

Next, a thicker layer of uncured silicone liquid 540 is poured over the coated wire 525/foil electrode 535 assembly and cured thermally. As layer 540 cures, it bonds permanently with the underlying thin silicone layer 510. The entire cuff assembly 550 may then be released from the substrate base 500 by dissolving mold release layer 505.

In the next step a plastic or metal sheet 600 is patterned by machining, said patterns being complementary to the desired pinch-hinge structure. Patterned sheet 600 serves as a master mold for fabricating the pinch-hinge structure. Mold 600 may next be coated with a mold release chemical 605. Subsequently, uncured silicone liquid 610 is poured over the mold 600 and allowed to settle into the patterned features.

Next, the cuff assembly 550 may be inverted and placed on top of uncured silicone 610. The entire set-up is then thermally cured such that the uncured silicone 610 permanently bonds with the cured silicone of the cuff assembly 550. The entire nerve cuff assembly 615 may be released from mold 600 by dissolving mold release chemical layer 605.

Longitudinal slit 625 may be defined by making a slit or cut on nerve cuff assembly 615 such that the depth of slit 625 is such that it reaches mold release chemical layer 520 located around metal wire 515. Slit opening 625 may then be used to introduce a solvent to enter and dissolve mold release chemical layer 520, thereby allowing the removal of metal wire 515 to form aperture 620. Finally, any extra silicone may be trimmed from nerve cuff assembly 615.

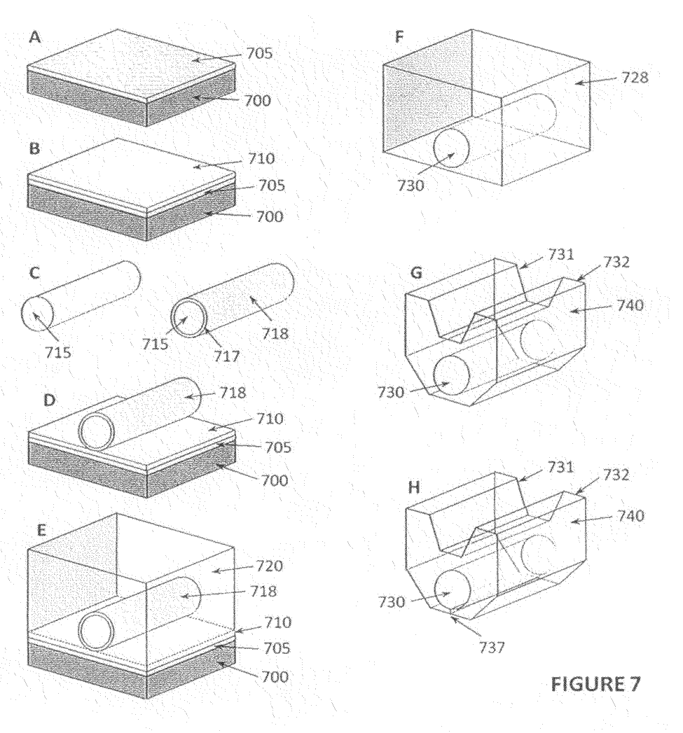

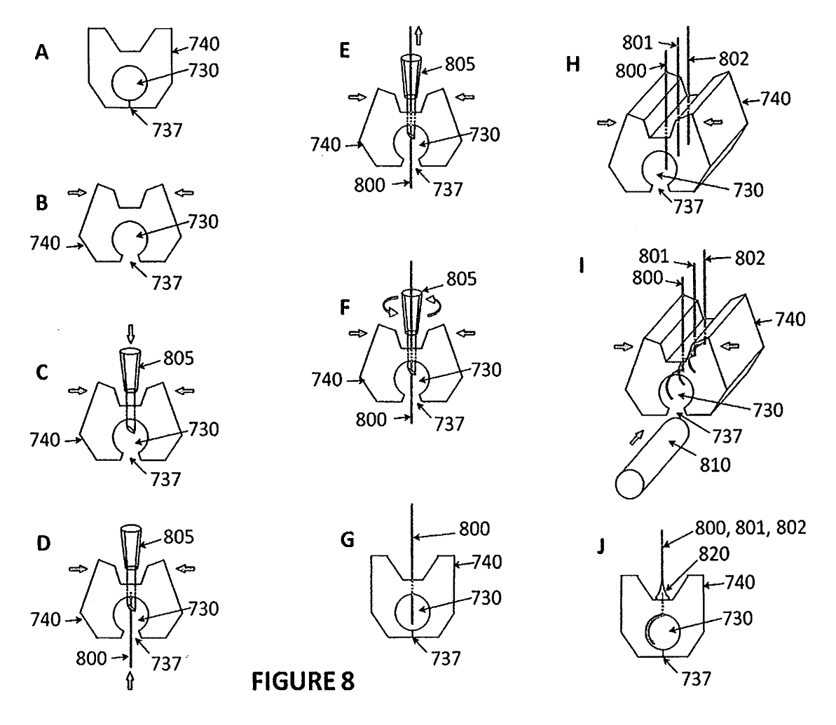

An alternative multi-step process for fabricating the clip-on micro-cuff electrodes is illustrated in FIGS. 7 and 8. In the first step, a thin flat sheet of plastic or metal is cleaned to be used as the base substrate 700. A mold release layer 705 may be coated on the substrate. The coating process may be done by a spin-coating process, followed by curing the mold release layer at room temperature.

In the next step, a thin layer 710 of medical grade silicone pre-polymer is spin-coated on the solid mold release layer. This layer is cured at a temperature appropriate to the silicone used. In the next step, a metal wire 715 of a known diameter is dip-coated with a thin mold release layer 717. The wet, mold release layer may be solidified at room temperature. The coated metal wire 718 is placed in conformal contact with the silicone layer 710.

In the next step, a thicker layer 720 of silicone pre-polymer liquid is poured over the coated wire 718, followed by thermal curing. As this thicker layer 720 is cured, it bonds permanently with the underlying thin silicone layer 710, to form a single structure 728. Subsequently, the single silicone structure 728 is released from the substrate 700 by dissolving the mold release layer 705 in an appropriate solvent. The mold release layer 717 on the coated wire 718 is dissolved, allowing for the wire to be easily pulled out from the silicone structure 728. Alternately, the wire 715 may be pulled out from the silicone structure 728 without dissolving the mold release layer 717 on the coated wire 718. The removal of the wire 715 leaves a cylindrical void 730 in the silicone structure 728. This cylindrical void 730 functions as the cuff.

In the next step, a sharp cutting tool is used to trim the free-standing silicone structure 728 with the hollow cylinder 730 to the desired lateral dimensions and shape of the clip-on micro-cuff device. The cutting tool also forms a pinch-hinge feature with two arms 731 and 732 by removing some silicone material in the structure 728. This step results in the main body 740 of the clip-on micro-cuff device. In the next step, a cutting tool is used to make a slit on the main body 740 in order to define the longitudinal opening 737 of the clip-on micro-cuff device. At this juncture of the fabrication process, the main cuff body 740 is ready to receive the electrodes. The cuff structure 730 is in a naturally closed position, with the flaps of the longitudinal opening 737 in contact with each other. Depressing the pinch-hinge structure allows the cuff 730 to be opened with an enlargement of the longitudinal opening 737.

To complete the fabrication of the clip-on micro-cuff electrode device, electrodes have to be incorporated in the main body 740 realized in the previous steps. A segment of an insulated conducting wire 800 is stripped off its insulation at one end to expose a short length of un-insulated wire. This bare part of the wire will function as an electrode lead inside the cylindrical cuff 730 of the device. The cuff 730 is slightly opened by holding the arms 731 and 732 of the pinch-hinge and applying a squeezing force to enlarge the longitudinal opening 737. A syringe needle 805 of a specific inner diameter is employed to pierce the cuff 730 from the pinch-hinge side of the main body 740. The needle 805 is allowed to exit from the cuff 730, through the enlarged longitudinal opening 737. In the next step, the insulated end of the electrode wire 800 is inserted into the syringe needle 805 from the open cuff side. When the wire 800 appears at the other side of the needle 805, it is held by tweezers and gently pulled through till a few cm of the wire comes out. In the next step, the syringe needle 805 is disengaged from the main body 740, leaving the wire 800 inside the cuff 730 and main body 740. This may be done by holding the electrode wire 800 at the cuff opening side and the main body 740, and gently pulling out the needle 805 with a twisting outward force. As the syringe needle 805 is pulled out, the material of the main body 740 collapses around the wire 800, holding it tightly. In the next step, the wire 800 is pulled further from the pinch-hinge side till only the un-insulated part of the wire 800 is inside the cylindrical cuff 730. The wire incorporation process may be repeated to introduce more electrodes as desired. FIG. 8 specifically shows two more electrode leads 801, 802 introduced into the cylindrical cuff 730 according to an embodiment of the present invention. At this juncture, the un-insulated parts of the electrode leads 800, 801, 802 are in the open space of the cuff cylinder 730. The insulated parts of the three electrode wires 800, 801, and 802 exit the main body 740 from the pinch hinge side, between the arms 731 and 732. The electrode leads 800, 801, 802 inside the cuff 730 have to be bent against its inner walls, so that the electrode leads lie along part of the circumference of the cuff 730. Therefore, in the next step, the cuff 730 is opened by depressing the arms 731 and 732 to enclose a pin 810 in the cuff 730. The pin 810 is rotated to bend the electrode leads 800, 801, and 802 so that they lie along the inner wall of cuff 730. Subsequently, the pin 810 is removed from the cuff 730. In the next step, the point of exit of the electrode wires 800, 801, 802 from the main body 740 or the interface electrode wire-cuff body may be reinforced by placing some silicone 820 pre-polymer and curing it thermally.

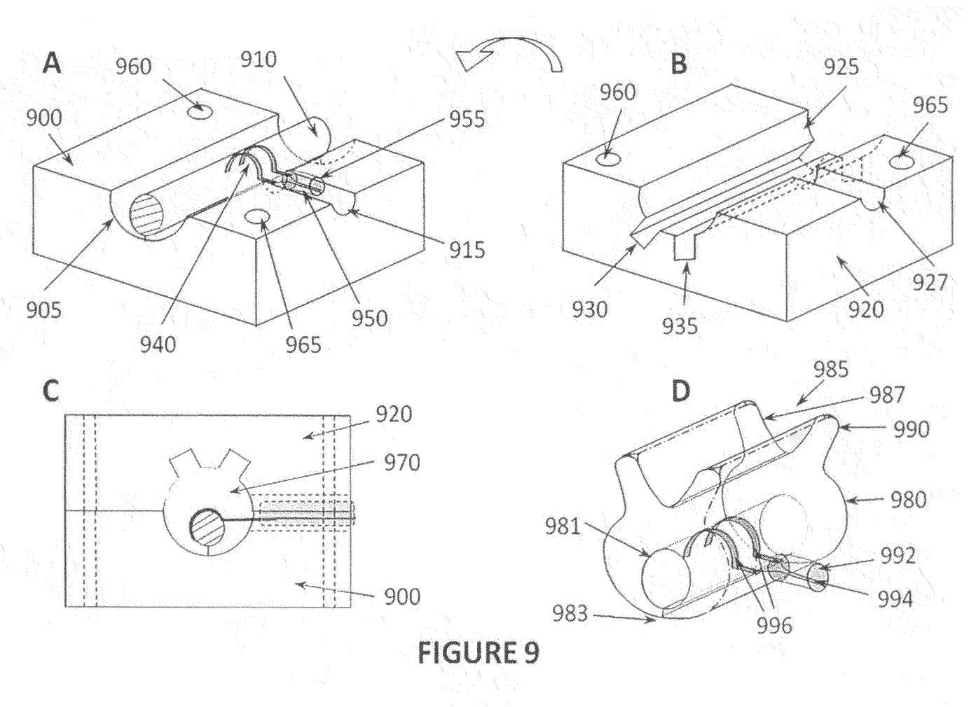

Another alternate technique for fabricating the clip-on micro-cuff electrode is by a single molding step using a master mold consisting of two or more individual parts, as illustrated in FIG. 9. The master mold parts may be made by machining plastic or metal, and assembled together. FIG. 9 shows a master mold made of plastic or metal with two parts. One part 900 of the master mold consists of a half cylinder 905, with a pin 910 attached at its bottom. A semi-circular groove 915 is provided on the part 900, extending from the centrally located pin to the outer edge of the part 900. This groove serves to hold electrode wires. Another part 920 of the master mold consists of a half cylinder 925, with two grooves 930 and 935 at its bottom. The two parts 900 and 920 of the master mold may be coated with a layer of mold release chemical by a dip-coating process. The mold release coating is allowed to cure at room temperature. Metal foil electrodes 940 (two are shown in FIG. 9A) may be placed in contact with approximately half the circumference of the pin 910. The number of metal foil electrodes depends on the application. In certain embodiments, the metal foil electrodes 940 may be previously cut to size from commercial available materials such as platinum, platinum-iridium, stainless steel or any other metal of choice. The metal foil electrodes 940 may be spot-welded to wires 950 made of stainless steel or any conducting material, which may serve as interconnecting wires from the implanted electrodes 940 to other electronic equipment. The stainless steel wires 950 may be coated by an insulating material and/or surrounded by an insulation layer 955. The wires 950 with the insulation 955 are placed in the groove leading away from the foil electrodes 940 placed on the pin 910.

The mold parts 900 and 920 are clamped together by means of holes 960 and 965 provided for clamping. Optionally, more holes, threaded or plain, can be employed to hold the parts of the mold together. After the mold parts 900 and 920 are clamped together, the master mold is placed vertically on the glass Petri dish and silicone pre-polymer liquid is poured into the enclosed cavity 970 of the mold. The silicone is cured thermally at a temperature that depends on the specific silicone elastomer used. Following the curing process, the master mold assembly is un-clamped and the parts 900 and 920 are detached. Dissolving the mold release layer in an appropriate solvent helps in the detachment of the parts 900 and 920. The removal of the mold release layer causes the cured silicone structure to be released from the mold. Subsequently, the cured silicone structure may be optionally trimmed to obtain the clip-on micro-cuff with the desired cuff length.

FIG. 9D shows an isometric view of the clip-on micro-cuff electrode 980 resulting from a single molding step. This micro-cuff incorporates a pinch hinge feature 985 according to an exemplary embodiment of the present invention. The cuff device 980 may include an aperture 981 having a longitudinal slit 983 opposite the pinch hinge 985, which includes a first arm 987 and a second arm 990. One or more electrodes 996 may be positioned in the inner circumference of the aperture 981 such that the electrodes 996 are positioned near or in direct contact with a nervous tissue when the cuff is positioned about a nerve and allowed to return to its normally closed position such that the aperture 981 completely encircles the nerve. A port 992 allows access to the electrodes 996 by one or more electrode wires 994, which may be spot welded to the electrodes 996. In some other embodiments, the port 992 may be located between the two arms 987 and 990 of the pinch hinge 985. In some other embodiments, the wires may be embedded directly into the body 980 without use of a port. The electrode wires 994, which may be either coated with insulation or without insulation, may also be further insulated from each other and the surrounding environment by silicone tubing or other appropriate insulating material, which may define port 992.

Upon installation, the clip-on structure of the nerve cuff may allow the electrodes embedded into the polymeric cuff wall to make proper electrical contact with the enclosed nervous tissue while reducing the possibility of causing trauma to the tissue. According to embodiments of the present invention, the pinch hinge design, which may allow the cuff to remain naturally closed, and the embedded electrode design feature, may allow the developed micro-cuff to conveniently record action potentials from the stimulated nerves or bio-potentials from any specific target tissue with significantly reduced electrical interference or cross-talk from the nearby tissues, varying amounts of extracellular fluids surrounding the nerve as would be present with the use of a partially open cuff, or other nearby physiological ambient.

Embodiments of the present invention apply to, but are not limited to, use in the research and treatment of obesity. Globally, over one billion people are morbidly obese. Bariatric surgery, currently the most effective treatment for morbid obesity, is not popular due to its highly invasive, non-reversible nature and expense. In contrast, FES based treatment is minimally invasive, reversible, and requires minimal post-operative care. Current FES-based obesity treatment research involves the electrical stimulation of sympathetic nerves and nerve fibers, such as those associated with the greater splanchnic and celiac ganglion. Because embodiments of the present invention allow for the relatively simple fabrication of micro-scale nerve cuffs, the present invention may be used in ongoing obesity treatment research utilizing small animal test subjects, such as rodents.

Potential applications for embodiments of the present invention are not limited to obesity research and treatment. Further applications include, but are not limited to, neuromuscular control, optical prostheses, and pain therapy. Other potential applications may address FES of smaller nerves, such as those of animals, children, or smaller peripheral nerves. While the embodiments describe above reference various implementations and exploitations, it will be understood that these embodiments and potential uses are illustrative and that the scope of the invention(s) is not limited to them. Many variations, modifications, additions, and improvements are possible. Further still, any steps described herein may be carried out in any desired order, and any desired steps may be added or deleted.

What is claimed is:

1. A method for fabricating a nerve cuff, comprising:
depositing a commercially available mold release chemical onto a substrate via spin coating to form a mold release layer;
depositing a first layer of silicone onto the mold release layer via spin coating and thermally curing the silicone layer;
coating a metal wire with a mold release chemical and allowing the chemical to solidify;
placing the coated metal wire in conformal contact with the thin layer of silicone;
placing at least one metal foil electrode in contact with approximately half the circumference of the metal wire;
depositing a second layer of uncured silicone over the wire-foil assembly and thermally curing the layer, wherein the second silicon layer is thicker than the first silicone layer;
dissolving the mold release layer on the substrate to release the structure from the substrate;
coating a mold configured to yield a pinch hinge structure with a mold release chemical layer and allowing the layer to solidify;
pouring uncured silicone liquid over the mold;
inverting the free-standing structure and placing it on the mold containing uncured silicone;
thermally curing the entire assembly such that the free-standing structure bonds with the uncured silicone;
dissolving the mold release layer on the mold to release the assembly from the mold;
making a longitudinal slit in the side of the assembly opposite the pinch hinge such that the depth of the slit reaches the coated metal wire;
dissolving the mold release chemical coating around the metal wire; and
removing the metal wire from the assembly.

2. The method of claim 1, further comprising the step of trimming the silicone body to obtain proper structure of the clip-on micro-cuff.

3. The method of claim 1, wherein the substrate is at least one of a plastic and a metal.

4. The method of claim 1, wherein the silicone is medical grade silicone.

5. The method of claim 1, wherein the metal foil is a conductive material.

6. The method of claim 5, wherein the conductive material is at least one of platinum, platinum-iridium, and stainless steel.

7. The method of claim 1, wherein the metal foil is spot-welded to conducting wires.

8. The method of claim 7, wherein the conducting wires are stainless steel wires.

9. The method of claim 7, wherein the conducting wires are insulated.

10. The method of claim 9, wherein the conducting wires are further insulated by medical grade silicone tubing.

11. The method of claim 1, wherein at least one of a biocompatible polyimide, poly ethylene, and a biocompatible injection moldable polymer is substituted for silicone.

12. A method for fabricating a nerve cuff, comprising:
depositing a commercially available mold release chemical onto a substrate via spin coating to form a mold release layer;
depositing a first layer of silicone onto the mold release layer via spin coating and thermally curing the silicone layer;
coating a metal wire with a mold release chemical and allowing the chemical to solidify;
placing the coated metal wire in conformal contact with the thin layer of silicone;
depositing a second layer of uncured silicone over the wire-foil assembly and thermally curing the layer, wherein the second silicon layer is thicker than the first silicone layer;
dissolving the mold release layer on the substrate to release the silicone structure from the substrate;
dissolving the mold release coating on the metal wire to yield a hollow cylindrical cuff in the silicone structure;
forming the desired shape and dimensions of the nerve cuff features including the pinch hinge on the silicone structure;
cutting a longitudinal slit on the cylindrical cuff opposite the pinch hinge in the silicone structure;
inserting electrode wires into the cuff via a syringe needle, such that un-insulated parts of the wires serve the function of electrode leads inside the cuff, and the segments of the wires outside the cuff are insulated;
holding the electrode wires in place by the cuff's silicone material after withdrawal of the needle; and
reinforcing the point of exit of the electrode wires from the main body of the cuff device.

13. The method of claim 12, wherein the reinforcing is done by at least one of silicone, silicon epoxy, and any other reinforcing material.

14. The method of claim 12, wherein the substrate is at least one of a plastic and a metal.

15. The method of claim 12, wherein the silicone is medical grade silicone.

16. The method of claim 12, wherein the metal foil is a conductive material.

17. The method of claim 16, wherein the conductive material is at least one of platinum, platinum-iridium, and stainless steel.

18. The method of claim 12, wherein the metal foil is spot-welded to conducting wires.

19. The method of claim 18, wherein the conducting wires are stainless steel wires.

20. The method of claim 18, wherein the conducting wires are insulated.

21. The method of claim 20, wherein the conducting wires are further insulated by medical grade silicone tubing.

22. The method of claim 12, wherein at least one of a biocompatible polyimide, poly ethylene, and a biocompatible injection moldable polymer is substituted for silicone.

23. A method for fabricating a nerve cuff, comprising:
making a master mold of plastic or metal consisting of two parts;
machining the first part of the master mold to define a half cylinder wherein a pin with a suitable cross-section is placed at the bottom of the half cylinder;
machining the first part to define a groove perpendicular to the half cylinder;
machining the second part of the master mold to define a half cylinder wherein two grooves are made to form the two arms of the pinch hinge;
coating both parts of the master mold by a mold release chemical layer and allowing the chemical to solidify;
placing at least one metal foil electrode in contact with approximately half the circumference of the cylindrical pin in the first part of the mold;
placing the second part of the master mold over the first part and securing them together via suitable clamps;
placing the master mold assembly vertically on a base and pouring silicone pre-polymer into the cavity of the mold assembly;
thermally curing the silicone inside the cavity of the master mold;
separating the two parts of the master mold and dissolving the mold release coating in an appropriate solvent;
releasing the cured silicone structure.

24. The method of claim 23, further comprising the step of trimming the cured silicone structure to obtain a nerve cuff of the desired shape and size.

25. The method of claim 23, wherein the base is a glass plate.

26. The method of claim 23, wherein the master mold material is at least one of a plastic and a metal.

27. The method of claim 23, wherein the silicone is medical grade silicone.

28. The method of claim 23, wherein the metal foil is a conductive material.

29. The method of claim 28, wherein the conductive material is at least one of platinum, platinum-iridium, and stainless steel.

30. The method of claim 23, wherein the metal foil is spot-welded to conducting wires.

31. The method of claim 30, wherein the conducting wires are stainless steel wires.

32. The method of claim 30, wherein the conducting wires are insulated.

33. The method of claim 32, wherein the conducting wires are further insulated by medical grade silicon tubing.

34. The method of claim 23, wherein at least one of a biocompatible polyimide, poly ethylene, and a biocompatible injection moldable polymer is substituted for silicone.

35. A nerve cuff, comprising:
a member including an aperture having a longitudinal slit;
a pinch hinge having a first arm and a second arm, the first arm spaced apart from the second arm, the first and second arms attached to the member;
at least one electrode positioned in the member;
wherein the longitudinal slit is configured to open when a compressive force is applied to an outer surface of the first arm and an outer surface of the second arm, whereby the first arm and second arm are squeezed together.

36. The nerve cuff of claim 35, wherein the member is configured such that the spring tension of the member holds the longitudinal slit in a normally closed position.

37. The nerve cuff of claim 35, wherein the nerve cuff is configured for use on micro-scale diameter nerves.

38. The nerve cuff of claim 35, wherein the member and pinch hinge include at least one of a biocompatible silicone elastomer, a biocompatible polyimide, poly ethylene, and a biocompatible injection moldable polymer.

39. The nerve cuff of claim 35, wherein the member and pinch hinge are a single piece.
